(19) United States
(12) Patent Application Publication
(10) Pub. No.: US 2019/0125657 A1
(43) Pub. Date: May 2, 2019

(54) COSMETIC USE OF KHAYA SENEGALENSIS EXTRACT

(71) Applicants: BASF Beauty Care Solutions France SAS, Lyon (FR); Centre National de la Recherche Scientifique, Paris (FR); Universite Claude Bernard Lyon I, Villeurbanne (FR)

(72) Inventors: Valérie ANDRE, Ampuis (FR); Nicolas BERTHELEMY, Pagy-sur-Moselle (FR); Isabelle BONNET, Lyon (FR); Sèbastien CADAU, L'Isle d'Abeau (FR); Christine JEANMAIRE, Nancy (FR); Philippe MOSER, Dommartemont (FR); Delphine RIVAL, Ternay (FR); Patricia ROUSSELLE, Lyon (FR)

(21) Appl. No.: 16/093,177

(22) PCT Filed: Apr. 11, 2017
(86) PCT No.: PCT/FR2017/050869

§ 371 Date: Oct. 12, 2018

(30) Foreign Application Priority Data
Apr. 12, 2016 (FR) 1653193

(51) Int. Cl. A61K 8/9789; A61Q 19/08; A61Q 7/00; A61Q 19/00; A61K 36/58

(52) U.S. Cl. CPC A61K 8/9789; A61Q 19/08; A61K 36/58; A61Q 19/008; A61Q 7/00

(57) ABSTRACT

The present invention relates to the cosmetic use of a Khaya senegalensis extract, obtained by aqueous extraction, for maintaining and/or increasing the expression of collagen XVIII in the skin and/or the mucosae and/or the scalp and/or the skin appendages, for maintaining and/or increasing the firmness and/or the elasticity of the skin and/or the mucosae and/or the scalp and/or for reducing the visibility of the skin's pores and/or making the skin smoother and/or for limiting and/or reducing perspiration and/or limiting and/or reducing loss of head hair and/or body hair and/or increasing the growth of head hair and/or body hair and/or reducing or limiting sebum production. Finally, the present invention relates to a cosmetic care method comprising the application of a Khaya senegalensis extract or of a cosmetic composition comprising same, for maintaining and/or increasing the expression of collagen XVIII in the skin and/or the mucosae and/or the scalp and/or the skin appendages, for maintaining and/or increasing the firmness and/or the elasticity of the skin and/or the mucosae and/or the scalp and/or for reducing the visibility of the skin's pores and/or making the skin smoother and/or for limiting and/or reducing perspiration and/or limiting and/or reducing loss of head hair and/or body hair and/or increasing the growth of head hair and/or body hair and/or reducing or limiting sebum production.

The present invention relates to a novel cosmetic and/or dermatological ingredient and the cosmetic and/or dermatological use thereof and also a cosmetic composition comprising same, for maintaining and/or increasing the expression of collagen XVIII in the skin and/or the mucous membranes and/or the scalp and/or the skin appendages, in particular at the basal laminae, and thus maintaining and/or increasing the firmness and/or elasticity of the skin and/or the mucous membranes and/or the scalp and/or for decreasing the visibility of cutaneous pores and/or making the skin smoother and/or for limiting and/or reducing perspiration and/or limiting and/or reducing the loss of head hair and/or body hair and/or increasing the growth of head hair and/or body hair and/or reducing or limiting the production of sebum.

The basal laminae, also referred to as basal membranes, have a support and cohesion function for the different compartments of the skin, in particular at the epithelium, especially the epidermis, dermis, adipocytes and endothelial cells. Degradation of the basal laminae, especially the dermoepidermal junction (DEJ), is observed over the course of aging of the skin and is reflected in a loss of the structure and properties of the various compartments. Thus, degradation of the dermoepidermal junction is reflected in the dermis by a loss of firmness and elasticity, in the epidermis by a decrease in the thickness of the epidermis, and in the sebaceous glands by an increase in the visibility of cutaneous pores, due in particular to an increase in their diameter.

It is known that collagens play an important role at the basal laminae, especially of the dermoepidermal junction. This is the case in particular for type XVIII collagen.

Collagen XVIII is a collagen of the family of the heparan sulfate proteoglycans, present in all the basal laminae of the body and synthesized by keratinocytes, adipocytes, epithelial cells of the sudoriparous glands, stem cells of hair follicles and endothelial cells. It is also the only collagen with heparan sulfate that is present in all the compartments of the skin.

It plays an important structural role at these basal laminae, especially the DEJ, the endothelial and epithelial basal laminae and adipocytes. Thus, the decreased expression of collagen XVIII, especially over the course of aging of the skin as is demonstrated in example 2, and/or the degradation thereof, leads to deterioration of the basal lamina, disorganization of the architecture of the skin and the abovementioned manifestations thereof. Thus, the decreased expression of collagen XVIII leads to collapse of the dermoepidermal junction and a decrease in exchanges between the dermis and the epidermis, a loss of firmness and a loss of elasticity of the skin.

At the DEJ of the sebaceous glands, degradation of and/or decreased expression of collagen XVIII induces collapse of the cutaneous pore and an increase in the opening diameter of the pore.

At the basal lamina of the sudoriparous gland, degradation of and/or decreased expression of collagen XVIII induces collapse of the sudoriparous gland and also an increase in the opening thereof, and therefore an increase in perspiration. At the basal lamina of the hair follicle, degradation of expression of collagen XVIII induces a drop in nutrient exchange and therefore a decrease in the structure and metabolism of the hair bulb and a loss of body hair and/or head hair. In addition, the collagen XVIII protein is located at basal laminae around adipocytes. Thus, the degradation and/or decreased expression thereof induces destructuring of the adipocytes, which leads to a loss of support and hence of firmness of the skin.

At the endothelial cells, the degradation of and/or decreased expression of collagen XVIII induces a decrease in the supply of nutrients, inducing at the dermis a drop in the synthesis of the structural molecules of the dermis and therefore a loss of firmness and elasticity.

The essential structural role of collagen XVIII at the basal laminae, in particular the dermoepidermal junction, but also the basal laminae of adipocytes, endothelial cells, sudoriparous glands and hair follicles, make it a target of interest in the fields of cosmetics and dermatology.

Surprisingly, the inventors have discovered that an extract of the plant Khaya senegalensis has the ability to increase the expression of collagen XVIII at the skin and/or the mucous membranes and/or the scalp and/or the skin appendages, in particular at the dermoepidermal junction, thereby making it possible to increase both the firmness and/or elasticity of the skin and/or the mucous membranes and/or the scalp, and also to decrease the visibility of the pores, that is to say decrease the opening thereof, and/or to make skin smoother and/or to limit and/or reduce perspiration and/or to limit and/or reduce the loss of head hair and/or body hair and/or increase the growth of head hair and/or body hair and/or reduce or limit the production of sebum.

Khaya senegalensis is a tree also referred to as cailcedrat, belonging to the Meliaceae family and found in several African countries, especially Benin, Cameroon, Senegal, Guinea, Ivory Coast or else Burkina Faso. It is also found in northern Australia and New Caledonia. The Khaya senegalensis bark according to the invention originates from Burkina Faso.

Khaya senegalensis is a plant used for the large-scale construction of dugout canoes in Africa. In addition, its seeds are rich in oleic acid, making therefrom an oil used in West African cuisine. Khaya senegalensis is moreover known as a medicinal plant. Thus, the seeds and leaves are used to combat fever and headaches. The bark in particular is known for its anti-inflammatory properties, and therefore is used in the treatment of arthritis, lumbago and other joint pains, and also in the treatment of dermatoses. Decoctions of bark of the plant have been used as disinfectants.

Patent application WO2012033634 discloses cosmetic anti-aging compositions which may be applied topically, comprising, among other plant extracts, a Khaya senegalensis extract for inhibiting synthesis of metalloproteases 1 (MMP1s) and stimulating the synthesis of procollagen I. However, this application does not disclose the cosmetic use of a Khaya senegalensis extract for maintaining and/or increasing the expression of collagen XVIII or for decreasing the visibility of cutaneous pores and/or making the skin smoother and/or for maintaining and/or increasing the firmness and/or elasticity of the skin and/or the mucous membranes and/or the scalp and/or for limiting and/or reducing perspiration and/or limiting and/or reducing the loss of head hair and/or body hair and/or increasing the growth of head hair and/or body hair and/or reducing or limiting the production of sebum. Moreover, this extract is not obtained by aqueous extraction.

Patent application EP1019016 also discloses a synergistic anti-aging complex intended especially for topical application to the skin and comprising a dehydrated extract of Khaya senegalensis bark, said complex making it possible to combat UV radiation, the inflammatory processes generated by this radiation, and to reduce lipid peroxidation of fibroblasts. This application does not disclose the use of a K. senegalensis extract for maintaining and/or increasing the expression of collagen XVIII or for decreasing the visibility of cutaneous pores and/or making the skin smoother and/or for maintaining and/or increasing the firmness and/or elasticity of the skin and/or the mucous membranes and/or the scalp and/or for limiting and/or reducing perspiration and/or limiting and/or reducing the loss of head hair and/or body hair and/or increasing the growth of head hair and/or body hair and/or reducing or limiting the production of sebum.

Application WO2011117642 also discloses hair care compositions comprising a K. senegalensis extract, said compositions being suitable for different uses including moisturization, anti-dandruff action or else volumizing effect. This patent application does not disclose any cosmetic use for the skin and/or the mucous membranes, nor for increasing the expression of collagen XVIII, nor for increasing the firmness or elasticity of the skin and/or the mucous membranes and/or the scalp, nor for decreasing the visibility of the pores, nor for limiting and/or reducing perspiration and/or limiting and/or reducing the loss of head hair and/or body hair and/or increasing the growth of head hair and/or body hair and/or reducing or limiting the production of sebum. Thus, to the applicants' knowledge, no document discloses the uses of the present invention.

The aim of the present invention is to provide an entirely novel cosmetic active ingredient capable of maintaining and/or increasing the expression of collagen XVIII at the skin and/or the mucous membranes and/or the scalp and/or the skin appendages, in particular the skin glands, especially sudoriparous glands and sebaceous glands, and hair follicles. Such an ingredient has the advantage of maintaining and/or increasing the firmness and elasticity of the skin and/or the mucous membranes and/or the scalp, but also of decreasing the visibility of the cutaneous pores, of making the skin smoother, of limiting and/or reducing perspiration and/or of limiting and/or reducing the loss of body hair and/or head hair and/or of increasing the growth of head hair and/or body hair and/or of reducing or limiting the production of sebum.

The advantage of this novel cosmetic active ingredient is that of being topically acceptable for the skin and/or the mucous membranes and/or the scalp, non-toxic, readily available, being able to be easily manufactured and packaged on an industrial scale.

A first subject of the present invention therefore relates to the cosmetic use of a Khaya senegalensis extract, preferentially obtained by aqueous extraction, for maintaining and/or increasing the expression of collagen XVIII in the skin and/or the mucous membranes and/or the scalp and/or the skin appendages, especially sudoriparous and sebaceous and hair follicles, in particular in the basal laminae.

Preferentially, the extract according to the invention maintains and/or increases the expression of collagen XVIII in the basal laminae, preferentially the epithelial basal lamina, preferentially the DEJ, including the DEJ of the skin, and/or the mucous membranes and/or the scalp and/or the sebaceous glands, and the basal laminae of adipocytes, endothelial cells, sudoriparous glands and/or hair follicles.

For the purpose of the present invention, “cosmetic use and/or cosmetic composition” is intended to mean a non-pharmaceutical composition and/or use, that is to say which does not require therapeutic treatment, that is to say intended for any “healthy” area of the skin and/or mucous membranes and/or scalp.

“Healthy area of the skin and/or mucous membranes and/or scalp” is intended to mean an area of the skin or mucous membranes or of the scalp to which the extract according to the invention is applied and which is referred to as “non-diseased” by a dermatologist, that is to say which does not have an infection, scar, skin disease or disorder such as candidiasis, impetigo, psoriasis, eczema, acne or dermatitis, or wounds or injuries and/or other dermatoses.

The extract according to the invention may be applied to all or part of the skin of the body and/or the face and/or the scalp, preferentially the legs, thighs, arms, stomach, bust, neck, armpits, lips, more preferentially still all or part of the face, preferentially the cheeks, forehead, chin, lips, area around the eyes, the “T” zone of the face.

There are several collagens, such as type I, III, IV, V, VI, VII, XII, XIII, XIV, XVI, XVII, XVIII, XXIV, XXIX collagen, present in the skin and/or the mucous membranes and/or the scalp. The invention relates to collagen XVIII.

“Mucous membrane” is intended to mean the ocular mucous membrane, the vaginal mucous membrane, the urogenital mucous membrane, the anal mucous membrane, the nasal mucous membrane and/or the oral, labial and/or gingival mucous membrane; preferentially, the ocular and/or oral mucous membranes.

“Expression of collagen XVIII” is intended to mean gene expression, that is to say the expression of mRNAs (messenger RNAs) and/or the protein expression of collagen XVIII. Preferentially, this is the protein expression of collagen. Preferentially this is the expression of collagen XVIII by keratinocytes, adipocytes, endothelial cells, epithelial cells of the sudoriparous glands and/or stem cells of hair follicles.

Preferentially, the collagen is human collagen, in particular of the human skin and/or mucous membranes and/or scalp. The expression of collagen XVIII may be measured according to conventional methods. The protein expression of collagen XVIII is preferentially measured on cells which produce it, that is to say keratinocytes, adipocytes, endothelial cells, epithelial cells of the sudoriparous glands and/or stem cells of the hair follicles.

Advantageously, the protein expression of collagen XVIII is measured on keratinocytes, adipocytes, endothelial cells, epithelial cells of the sudoriparous glands and/or stem cells of the hair follicles, in particular on keratinocytes, adipocytes and/or endothelial cells by in vitro measurement, preferentially by measurement by confocal microscopy following immunolabeling, for example according to the method as, presented in example 3.

For the purpose of the present invention, “immunolabeling” is intended to mean the technique consisting in fixing a fluorescent antibody specific to the collagen XVIII protein and quantifying the fluorescence by microscopy, preferentially confocal microscopy.

“Maintaining the expression of collagen” is intended to mean preventing a reduction in the level of gene and/or protein expression of collagen relative to the gene and/or protein expression detected in the absence of the extract according to the invention, especially the level of gene and/or protein expression of collagen that is observed over the course of the extrinsic or intrinsic aging of the skin and/or the mucous membranes and/or the scalp.

For the purpose of the present invention, “increasing the expression of collagen” is intended to mean an increase in the level of gene and/or protein expression of collagen, preferentially of at least 3% in the presence of the K. senegalensis extract, preferentially of at least 10%, more preferentially still of at least 20% relative to the gene and/or protein expression detected in the absence of the extract according to the invention. Preferentially, this is an increase in the protein expression of collagen XVIII.

In a preferential embodiment of the invention, this is an increase in the protein expression of collagen XVIII measured in “normal” human cells which produce it, that is to say non-diseased cells. Advantageously, said increase is measured in the presence of the K. senegalensis extract prepared according to example 1a).

According to an advantageous embodiment of the invention, the increase in protein expression of collagen XVIII is measured in normal human keratinocytes, in normal human adipocytes, in normal endothelial cells, in normal epithelial cells of the sudoriparous glands and/or in normal stem cells of the hair follicles, in particular in normal human keratinocytes, in normal adipocytes, and/or in normal endothelial cells, more advantageously still in the presence of the K. senegalensis extract prepared according to example 1a).

Unless indicated otherwise, “normal” cells, “normal” keratinocytes, “normal” adipocytes, are intended to mean cells that are not diseased.

Preferentially, the protein expression of collagen XVIII is measured by confocal microscopy after immunocytochemical labeling using an anti-collagen XVIII antibody, as described in example 2.

A subject of the present invention is also the use of a K. senegalensis extract according to the invention for maintaining and/or increasing gene and/or protein expression, preferentially protein expression, of collagen XVIII, for increasing the thickness of the dermoepidermal junction at the skin, the mucous membranes and/or the skin appendages.

“Increasing the thickness of the dermoepidermal junction” is intended here to mean increasing the density thereof and/or increasing exchanges between the dermis and the epidermis and improving the structure thereof.

A subject of the present invention is the cosmetic use of a K. senegalensis extract according to the invention for maintaining and/or increasing gene and/or protein expression, preferentially protein expression, of collagen XVIII, for decreasing the visibility of cutaneous pores and/or making the skin smoother and/or for maintaining and/or increasing the firmness and/or elasticity of the skin and/or the mucous membranes and/or the scalp and/or for limiting and/or reducing perspiration and/or limiting and/or reducing the loss of head hair and/or body hair and/or increasing the growth of head hair and/or body hair and/or reducing or limiting the production of sebum.

For the purpose of the present invention, “decreasing the visibility of cutaneous pores” is intended to mean tightening the cutaneous pores, that is to say decreasing the opening diameter of the pores, and/or the density and/or the area of the pores at the surface of the skin, and/or preventing dilation of the cutaneous pores. The extract according to the invention therefore makes it possible to decrease the opening and/or the density of the pores at the surface of the skin.

The visibility of the pores of the skin may be demonstrated in vivo by an evaluation referred to as “scoring” by a dermatologist on a predefined area after application of a composition comprising the extract according to the invention. It may also be demonstrated by an objective instrumental method by image analysis that makes it possible to extract and quantify specific parameters from high-resolution photographs, with cross-polarized light, of the face of volunteers taken before and after application of a composition comprising the extract according to the invention.

The density of the cutaneous pores may also be measured in vivo by imaging, especially by the fringe projection technique, by measuring the parameter referred to as curvature, under the conditions described in particular in example 5b).

In a preferred embodiment of the invention, the K. senegalensis extract according to the invention is in an effective amount for decreasing the visibility of the pores of the skin by at least 10%, preferentially at least 20%, after 28 days of application of a cream comprising the extract according to the invention; advantageously, the K. senegalensis extract is prepared under the conditions described in example 1a), preferentially formulated in the form of an active ingredient as described in example 1e).

Moreover, according to the present invention, “making the skin smoother” is intended to mean increasing the homogeneity of the surface of the skin, and decreasing the cutaneous microrelief.

Moreover, according to the present invention, “limiting and/or reducing perspiration” is intended to mean tightening the duct of the sudoriparous gland and/or reducing the opening diameter of the sudoriparous glands and thus decreasing the amount of sweat produced.

Moreover, “limiting and/or reducing the loss of head hair and/or body hair” is intended to mean decreasing the number of head hairs and/or body hairs in the telogen phase.

These effects may be measured by conventional measurement methods well known to those skilled in the art.

Moreover, according to the present invention, “increasing the growth of head hair and/or body hair” is intended to mean increasing the rate of growth of head hair and/or body hair, which may be measured according to conventionally used methods.

Moreover, according to the present invention, “reducing or limiting the production of sebum” is intended to mean preventing the increase in, or decreasing the amount of, sebum secreted by the sebaceous glands. This property may be measured according to conventional methods well known to those skilled in the art, such as, for example, quantitative analysis of a sebum marker such as squalene in patch-type samples (such as Sebutape™).

For the purpose of the present invention, “increasing the firmness and/or elasticity” of the skin and/or the mucous membranes and/or the scalp is intended to mean an increase, for esthetic purposes, respectively of the firmness and/or the elasticity of the skin and/or the mucous membranes and/or the scalp, which have lost firmness and/or elasticity particularly due to a decrease in the expression of collagen XVIII, more particularly still at the dermoepidermal junction.

“Maintaining the firmness and/or elasticity” of the skin and/or the mucous membranes and/or the scalp is intended to mean preventing sagging of the skin and/or the mucous membranes and/or the scalp, particularly due to a decrease in the expression of collagen XVIII, more particularly still at the dermoepidermal junction.

The firmness and/or elasticity of the skin and/or the mucous membranes and/or the scalp may be measured according to conventional methods known to those skilled in the art, especially by in vivo measurement using a cutometer, or else a Tonoderm™ or a DynaSKIN combined with a dermaTOP.

Thus, preferentially, the K. senegalensis extract increases the elasticity of the skin and/or the mucous membranes and/or the scalp by at least 0.5%, preferentially at least 1.5%, more preferentially still at least 4% and more advantageously still at least 8% in the presence of the extract according to the invention.

In one embodiment of the invention, the increase in the elasticity of the skin is measured on reconstructed skin via a method based on evaluation of the deformation of the skin under the effect of a controlled air stream, said deformation being measured by laser, under the conditions described in example 4a).

In another embodiment of the invention, the increase in the elasticity of the skin is measured under the conditions described in example 4b) on the same reconstructed skin, by measuring the viscoelastic behavior of the skins, reflected via the parameter (Y2−Ue)/Y2 (FIG. 1). When said parameter decreases, the elasticity of the skin increases, implying that the skin returns more easily to its initial state.

The elasticity may also be measured with a cutometer in its immediate and/or overall component, as described in example 5a).

The K. senegalensis extract according to the invention is a topically acceptable cosmetic and/or dermatological extract.

For the purpose of the present invention, “topically acceptable” is intended to mean an ingredient suitable for topical application that is non-toxic and non-irritant for the skin and/or the mucous membranes and/or the scalp, that does not induce an allergic response and that is not chemically unstable.

The extract according to the present invention may be used orally or topically. Advantageously, it is used topically. For the purpose of the present invention, “topically” is intended to mean the direct local application and/or spraying of an ingredient onto the surface of the skin and/or the mucous membranes and/or the scalp.

The extract according to the invention may be any extract of all or part of the plant Khaya senegalensis and especially selected from the root, bark, flower, seed, seedling, aerial parts, in particular the stem of the branches and/or the leaf, and mixtures thereof. The extract according to the invention is preferentially a bark extract.

The extract may be obtained by plant extraction methods known in the field, for example by maceration of at least a part of the plant, preferably between 1% and 30% (w/w) by weight, preferentially between 10% and 20% (w/w) by weight, relative to the total weight of the part of the plant and of the solvent, in a solvent or a mixture of solvents such as water, alcohol, polyol, glycol, water/alcohol, water/glycol or water/polyol mixture from 100/0 to 0/100 (v/v). For example, the extract may be able to be obtained by extraction in a water/ethanol mixture, particularly in the respective proportions of 70/30 (v/v). Preferentially, the extract is, obtained by aqueous extraction.

The extract according to the invention may therefore be obtained by extraction of an amount of 10% to 20% by weight of bark relative to the total weight of bark and of solvent, preferentially water as sole solvent.

For the purpose of the present invention, “extract obtained by aqueous extraction” is intended to mean any extract obtained by extraction with an aqueous solution containing more than 60% by weight, advantageously at least 70% by weight, in particular at least 80% by weight, more particularly at least 90% by weight, particularly at least 95% by weight, of water relative to the total weight of the aqueous solution, even more advantageously not containing butylene glycol, in particular not containing alcohol, particularly only containing water.

The extract may be obtained by extraction at a temperature ranging from 4° C. to 95° C., preferentially from 20° C. to 95° C., more preferentially still from 50 to 95° C., preferably from 70° C. to 90° C. In a preferential embodiment, the extraction is carried out at 85° C.

The extraction may be carried out during a period of 1 hour to 24 hours, preferentially from 1 hour to 12 hours, more preferentially still from 1 hour to 6 hours. Advantageously, the extraction is carried out during a period of 1 hour.

In an advantageous embodiment of the invention, the extraction is carried out in 2 successive extraction temperature phases, a 1st extraction phase at a temperature ranging from 4° C. to 95° C., preferentially ranging from 20° C. to 70° C., advantageously at a temperature of 50° C., for a period from 1 hour to 12 hours, preferentially for a period of 4 hours, followed by a second extraction phase at a temperature ranging from 50° C. to 95° C., preferentially 85° C., for a period of 1 hour.

The aqueous extract may be decolored by any means, especially using activated carbon or decolorizing earths, in which case the process generally comprises an additional step of readjustment of the pH of the extract. As a variant or in addition, the process described above may comprise a step of deodorizing the extract according to any technique known to those skilled in the art, before or after the decoloring step.

According to one embodiment, the optionally decolored and/or deodorized extract may then be concentrated by evaporation or dehydrated by any means, especially by spray-drying or lyophilization. An adjuvant, preferentially maltodextrin, may be added before or after drying and preferentially before dehydration, to the liquid extract, in proportions ranging from 20 to 80% by weight of maltodextrin relative to the total weight of dry extract obtained after dehydration.

Thus, in a particularly advantageous embodiment of the invention, the extract is obtained by extraction, in water as sole solvent, of an amount of 20% by weight of Khaya senegalensis bark relative to the total weight of bark and water, at a temperature of 50° C. for a period of 4 hours, then at a temperature of 85° C. for a period of 1 hour. The extract thus obtained is then centrifuged and the pellet eliminated. The extract is then cooled to a temperature of 4° C., filtered, and maltodextrin is then added. The extract is sterilized (UHT) then spray-dried under the conditions described in example 1a).

In another embodiment, the extract according to the invention is obtained by extraction, in water as sole solvent, of an amount of 10% by weight of Khaya senegalensis bark relative to the total weight of bark and water, at a temperature of 50° C. for a period of 4 hours, then at a temperature of 85° C. during a period of 1 hour. The extract is centrifuged, the pellet eliminated, then the extract is cooled to a temperature of 4° C. The liquid extract obtained is filtered and maltodextrin is added. The extract is sterilized (UHT) then spray-dried under the conditions described in example 1b).

In yet another embodiment of the invention, the extract is obtained by extraction, in water as sole solvent, of an amount of 10% by weight of leaves of the plant relative to the total weight of leaves and water, at a temperature of 50° C. during a period of 4 hours, then at a temperature of 85° C. for a period of 1 hour. The extract is centrifuged, then cooled to a temperature of 4° C. The liquid extract obtained is dried and maltodextrin is added. The extract is sterilized (UHT) then spray-dried under the conditions described in example 1c).

In another embodiment, the extract according to the invention is obtained by extraction, in a water/ethanol mixture (70/30; v/v), of an amount of 20% by weight of Khaya senegalensis bark relative to the total weight of bark and solvent mixture, at a temperature of 85° C. during a period of 1 hour. The extract is centrifuged and the pellet eliminated. The extract is dried and maltodextrin added. The extract is spray-dried under the conditions described in example 1d).

In yet another embodiment, the extract may be obtained under the conditions described in example 1a) then diluted in a mixture of water and glycerin, in a final amount of 1.5% by weight relative to the total weight of extract, water and glycerin, under the conditions described in example 1e).

The K. senegalensis extract may be used alone as cosmetic and/or dermatological active ingredient or included within a cosmetic and/or dermatological composition. When it is used alone in the form of an active ingredient, the extract according to the invention is preferentially soluble and dissolved in a solvent, especially a polar solvent, such as water, an alcohol, a polyol, a glycol, or a mixture thereof, in the presence or absence of glycerin. Preferentially, the extract is dissolved in a mixture of water and glycerin to produce a cosmetic ingredient that can be easily formulated.

Advantageously, in this case, the extract is produced according to the protocol described in example 1e).

Therefore, another subject of the present invention is the topical use of the extract according to the invention on the skin and/or the mucous membranes and/or the scalp, alone or included within a cosmetic composition comprising at least one cosmetically acceptable excipient.

The K. senegalensis extract according to the invention is preferably present in the composition at a concentration from 1×10⁻⁴% to 10% by weight, preferentially between 1×10⁻⁴% and 5% by weight, more advantageously still between 1×10⁻³% and 3% by weight, relative to the total weight of the composition, in particular between 0.001 and 0.1% by weight relative to the total weight of the composition.

In one embodiment of the invention, the cosmetic composition containing the extract according to the invention is used for maintaining and/or increasing gene and/or protein expression, preferentially protein expression, of collagen XVIII in the skin and/or the mucous membranes and/or the scalp and/or the skin appendages, in particular the skin glands, especially sudoriparous glands and sebaceous glands, and hair follicles, in particular in the basal laminae, preferentially the DEJ, in particular of the skin and/or the mucous membranes and/or the scalp and/or the sebaceous glands, and/or the basal laminae of adipocytes, endothelial cells, sudoriparous glands and/or hair follicles, preferentially for increasing the thickness of the dermoepidermal junction at the skin and/or the mucous membranes and/or the scalp, for maintaining and/or increasing firmness and/or elasticity of the skin and/or the mucous membranes and/or the scalp, and/or for decreasing the visibility of cutaneous pores and/or making the skin smoother and/or for limiting and/or reducing perspiration and/or for limiting and/or reducing the loss of head hair and/or body hair and/or increasing the growth of head hair and/or body hair and/or reducing or limiting the production of sebum.

The cosmetic composition may also include one or more cosmetically acceptable excipients chosen from surfactants and/or emulsifiers, preservatives, buffers, chelating agents, denaturing agents, opacifiers, pH adjusters, reducing agents, stabilizers, thickeners, gelling agents, film-forming polymers, fillers, mattifying agents, gloss agents, pigments, dyes, fragrances and mixtures thereof. The CTFA (Cosmetic Ingredient Handbook, Second Edition (1992)) describes various cosmetic excipients suitable for use in the present invention.

Advantageously, the excipient(s) are chosen from the group comprising polyglycerols, esters, cellulose polymers and derivatives, lanolin derivatives, phospholipids, lactoferrins, lactoperoxidases, sucrose-based stabilizers, vitamin E and derivatives thereof, xanthan gums, natural and synthetic waxes, vegetable oils, triglycerides, unsaponifiable substances, phytosterols, silicones, protein hydrolyzates, betaines, amine oxides, plant extracts, sucrose esters, titanium dioxides, glycines and parabens, and more preferably from the group consisting of steareth-2, steareth-21, glycol-15 stearyl ether, cetearyl alcohol, phenoxyethanol, methylparaben, ethylparaben, propylparaben, butylparaben, butylene glycol, caprylyl glycol, natural tocopherols, glycerin, sodium dihydroxycetyl phosphate, isopropyl hydroxycetyl ether, glycol stearate, triisononanoin, octyl cocoate, polyacrylamide, isoparaffin, laureth-7, a carbomer, propylene glycol, hexylene glycol, glycerol, bisabolol, a dimethicone, sodium hydroxide, PEG 30-dipolyhydroxystearate, capric/caprylic triglycerides, cetearyl octanoate, dibutyl adipate, grapeseed oil, jojoba oil, magnesium sulfate, EDTA, a cyclomethicone, xanthan gum, citric acid, sodium lauryl sulfate, mineral oils and waxes, isostearyl isostearate, propylene glycol dipelargonate, propylene glycol isostearate, PEG 8, beeswax, hydrogenated palm kernel oil glycerides, lanolin oil, sesame oil, cetyl lactate, lanolin alcohol, castor oil, titanium dioxide, lactose, sucrose, low-density polyethylene, an isotonic saline solution, and mixtures thereof.

The cosmetic composition according to the invention may be chosen from an aqueous or oily solution, a cream or an aqueous gel or an oily gel, especially a shower gel, a milk, an emulsion, a microemulsion or a nanoemulsion, which is especially oil-in-water or water-in-oil or multiple or silicone-based, a mask, a serum, a lotion, a liquid soap, a dermatological bar, an ointment, a foam, a patch, an anhydrous product, which is preferably liquid, or pasty or solid, for example in the form of makeup powders, a rod or a stick, in particular in the form of a lipstick.

Advantageously, it is a cream or a serum.

The composition used according to the invention may also contain cosmetic active ingredients leading to a complementary or synergistic effect, such as anti-aging active agents. Among these active agents, mention may be made of active agents that stimulate the synthesis of macromolecules of the dermis or prevent the degradation thereof, agents that stimulate keratinocyte proliferation, soothing agents, moisturizing agents, or else agents that act on regulating the size of pores and/or the opening thereof.

Among anti-aging active agents, mention may be made of:
an agent which stimulates fibronectin synthesis, in particular a corn extract, such an extract being especially sold by BASF Beauty Care Solutions France under the name Deliner™, and the palmitoyl pentapeptide sold by the company Sederma under the trade name Matrixil®;
an agent which stimulates the formation of elastic fibers, such as an Origanum majorana extract sold under the name Dermagenist™ by the applicant;
an agent which stimulates perlecan and dystoglycan expression in the extracellular matrix and/or in the epithelial basal membrane, for example a Polygonum bistorta extract sold under the name Perlaura™ by BASF Beauty Care Solutions France;
an agent which protects extracellular matrix fibroblast growth factor (FGF2) against degradation thereof and/or denaturation thereof, especially a Hibiscus abelmoscus extract as described in the patent application in the name of BASF Beauty Care Solutions France published under number FR0654316 and sold by BASF Beauty Care Solutions France under the name Linefactor™ and/or an agent which stimulates fibroblast growth, for example a fermented soybean extract containing peptides, known as Phytokine™ sold by BASF Beauty Care Solutions France and also described in patent application EP 1 119 344 B1 (Laboratoires Expanscience), and preferentially a combination of these two extracts;
an agent which stimulates laminin synthesis, in particular a biotechnology-modified malt extract, such an extract being especially sold by BASF Beauty Care Solutions France under the name Basaline™;
an agent which stimulates hyaluronane synthase 2 (HAS2) expression and/or activity, such as the plant extracts described in patent application FR 2 893 252 A1 and in particular an aqueous extract of Galanga (Alpinia galanga) and sold by BASF Beauty Care Solutions France under the name Hyalufix™;
an agent which stimulates lysyl oxidase-like (LOXL) synthesis, such as an extract of Geophila cordifolia and those described in patent application FR 2 855 968, and in particular a dill extract and sold by BASF Beauty Care Solutions France under the name Lys'lastine™;
an agent which stimulates intracellular ATP synthesis, especially an extract of the alga Laminaria digitata;
an active agent which stimulates glycosaminoglycan synthesis, such as the product of milk fermentation;
an active agent which stimulates collagen, such as retinol and/or vitamin C;
an active agent which inhibits metalloproteases (MMPs) such as more particularly MMPs 1, 2, 3 and 9, such as retinoids and derivatives, oligopeptides and lipopeptides, lipoamino acids, the extract of leaves of Argania spinosa sold by BASF Beauty Care Solutions France SAS under the name Arganyl™; lycopene; isoflavones, quercetin, kaempferol, apigenin,
a refilling agent, especially the hyaluronic acid filling spheres sold by BASF Beauty Care Solutions France under the name Hyaluronic Filling Spheres™,
an agent for increasing the expression of LOX, for enhancing the architecture of the epidermis, such as, for example, a Cichorium intybus extract, sold under the name LOX-AGE™ by BASF Beauty Care Solutions France,
an agent for increasing collagen deglycation and/or increasing the expression of type I collagen, such as a combination of an extract of Salvia miltiorrhiza leaves and of niacin, sold by BASF Beauty Care Solutions France under the name CollRepair™,
an agent which stimulates lumican and collagen synthesis, such as a synthetic acetyl Gln Asp Val His tetrapeptide sold by BASF Beauty Care Solutions France under the name Dermican™ and described in patent application WO2005/120554 A1,
an agent for protecting and stimulating elastin and collagen, such as the extract of Manilkara multinervis leaves sold by BASF Beauty Care Solutions France under the name Elestan™ and the extract of Eperua falcata root sold by BASF Beauty Care Solutions France under the name Eperuline™,
an anti-pigment-spots agent, especially acting by inhibiting melanin synthesis, such as the synergistic complex of Pisum sativum extract and sucrose dilaurate, sold by BASF Beauty Care Solutions France under the name Actiwhite™, or hydroxyphenoxy propionic acid sold by BASF Beauty Care Solutions France under the name Radianskin™.

The agents which stimulate keratinocyte proliferation, preferentially of use in the composition according to the invention, comprise especially retinoids such as retinol and esters thereof, including retinyl palmitate and phloroglucinol. The agents which stimulate keratinocyte differentiation comprise, for example, minerals such as calcium and lignans such as secoisolariciresinol and also the extract of Achillea millefollium sold under the name Neurobiox™ by BASF Beauty Care Solutions France.

As soothing agents preferentially of use in the composition according to the invention, mention may be made of: pentacyclic triterpenes, ursolic acid and salts thereof, oleanolic acid and salts thereof, betulinic acid and salts thereof, salicylic acid salts and in particular zinc salicylate, bisabolol, allantoin, omega-3 unsaturated oils, cortisone, hydrocortisone, indomethacin and betamethasone, anti-inflammatory active agents, and especially those described in application FR 2 847 267, in particular the Pueraria lobata root extract sold under the name Inhipase® by BASF Beauty Care Solutions France SAS, extracts of Theobroma cacao.

Among the agents acting on the regulation of pore size and/or the opening thereof and/or on sebum production, mention may be made by way of example of a Cichorium intybus extract sold under the name LOX-AGE™ by BASF Beauty Care Solutions France, or synthetic sarcosine sold under the name MatXS™ Clinical and/or an Orthosiphon stamineus extract as described in patent application WO2010/063674 in the name of BASF Beauty Care Solutions France and sold under the name MAT XS™ Bright.

As moisturizing agents preferentially of use in the composition according to the invention, mention may be made of: a combination of pullulan, of sodium hyaluronate and of sodium alginate, as sold by BASF Beauty Care Solutions France under the name Patch2O™.

Yet another object of the present invention is a cosmetic care process, characterized in that it comprises the application, preferably topically, of the K. senegalensis extract according to the invention or of a cosmetic composition comprising same, in particular at the skin and/or the mucous membranes and/or the scalp, for maintaining and/or increasing gene and/or protein expression, preferentially protein expression, of collagen XVIII in the skin and/or the mucous membranes and/or the scalp and/or the skin appendages, in particular in the basal laminae, preferentially the DEJ including the DEJ of the skin, and/or the mucous membranes and/or the scalp and/or the sebaceous glands, the basal laminae of adipocytes, endothelial cells, sudoriparous glands and/or hair follicles, for maintaining and/or increasing the firmness and/or elasticity of the skin and/or the mucous membranes and/or the scalp and/or for decreasing the visibility of cutaneous pores and/or making the skin smoother and/or limiting and/or reducing perspiration and/or limiting and/or reducing the loss of head hair and/or body hair and/or increasing the growth of head hair and/or body hair and/or reducing or limiting the production of sebum.

In one embodiment of the present invention, the care process consists of the topical application of the K. senegalensis extract according to the invention or of a cosmetic composition comprising same over all or part of the skin and/or the mucous membranes of the body and/or the face and/or the scalp, preferentially the legs, thighs, arms, stomach, bust, neck, armpits, lips, more preferentially still all or part of the face, and preferentially the cheeks, forehead, chin, lips, area around the eyes, the “T” zone of the face.

Thus, the cosmetic care process consists of the topical application of a cosmetic composition comprising the K. senegalensis extract according to the invention at a concentration from 1×10⁻⁴% to 10% by weight, preferentially from 1×10⁻⁴% to 5% by weight, more advantageously still from 1×10⁻³% to 3% by weight, in particular from 0.001% to 0.1% by weight relative to the total weight of the composition.

A final subject of the present invention relates to a K. senegalensis extract, optionally in the form of a pharmaceutical, preferentially dermatological composition, as described above, for its use, for preventing and/or treating diseases involving a loss of gene and/or protein expression, preferentially protein expression of type XVIII collagen, such as those involved in eye development, such as for example Knobloch syndrome, those involved in cerebral development, those involved in the nervous system and certain glomerulopathies.

The extract according to the invention may be in the form of a pharmaceutical, preferentially dermatological composition, comprising at least one pharmaceutically and/or dermatologically acceptable excipient.

In one embodiment of the invention, said composition is applied topically and/or orally, preferentially topically.

Advantageously, the K. senegalensis extract according to the invention is present in the pharmaceutical, preferentially dermatological, composition at a concentration from 1×10⁻⁴% to 10% by weight, preferentially between 1×10⁻⁴% and 5% by weight, more advantageously still between 1×10⁻³% and 3% by weight, relative to the total weight of the composition, in particular between 0.001% and 0.1% by weight relative to the total weight of the composition.

Examples referring to the description of the invention are presented below.

These examples are given for illustrative purposes and in no way limit the scope of the invention. Each of the examples has a general scope. The examples are an integral part of the present invention, and any feature appearing to be novel over any prior art whatsoever, from the description taken in its entirety, including the examples, is an integral part of the invention.

### LIST OF THE FIGURES

FIG. 1: Evaluation of the biomechanical properties of the skin, expression of the residual deformation of the skin and of the viscoelastic component.

FIG. 2: Coefficients of in vivo measurement of skin elasticity.

### EXAMPLE 1

Preparation of different Khaya senegalensis extracts

### Example 1a

An amount of 20% by weight of bark from the Khaya senegalensis plant, relative to the total weight of bark and of water, was milled then extracted in water as sole solvent at a temperature of 50° C. during a period of 4 hours, then at a temperature of 85° C. during a period of 1 hour. The extract was then centrifuged and the pellet was eliminated.

The extract was then cooled to a temperature of 4° C. The liquid extract obtained was filtered and maltodextrin was added (in an amount such that the maltodextrin represents 50% by weight relative to the total weight of the mixture of liquid extract and maltodextrin obtained after dehydration).

The whole mixture was UHT sterilized then spray-dried.

### Example 1b

An amount of 10% by weight of bark from the Khaya senegalensis plant, relative to the total weight of bark and of water, was milled then extracted in water as sole solvent at a temperature of 50° C. during a period of 4 hours, then at a temperature of 85° C. during a period of 1 hour. The extract was then centrifuged, then cooled to a temperature of 4° C. The liquid extract obtained was filtered, and maltodextrin was added. The extract was UHT sterilized then spray-dried.

### Example 1c

An amount of 10% by weight of leaves from the plant, relative to the total weight of leaves and of water, was milled then extracted in water as sole solvent at a temperature of 50° C. during a period of 4 hours, then at a temperature of 85° C. during a period of 1 hour. The extract was then centrifuged, then cooled to a temperature of 4° C. The liquid extract obtained was filtered, dried, and maltodextrin was added.

The extract was UHT sterilized then spray-dried.

### Example 1d

An amount of 20% by weight of bark from the Khaya senegalensis plant, relative to the total weight of bark and of the solvent mixture below was milled then extracted in a water/ethanol mixture (70/30; v/v) at a temperature of 85° C. during a period of 1 hour. The extract was then centrifuged and the pellet was eliminated. The maltodextrin was added. The extract was spray-dried.

### Example 1e

An amount of 20% by weight of bark from the Khaya senegalensis plant, relative to the total weight of bark and of water, was milled then extracted in water as sole solvent at a temperature of 50° C. during a period of 4 hours, then at a temperature of 85° C. during a period of 1 hour. The extract was then centrifuged and the pellet is eliminated.

The extract is then cooled to a temperature of 4° C. The liquid extract obtained was filtered and maltodextrin was added (in an amount such that the maltodextrin represents 50% by weight relative to the total weight of the mixture of liquid extract and maltodextrin obtained after dehydration).

The whole mixture was UHT sterilized then spray-dried. The extract is then formulated in the form of a cosmetic active ingredient as follows: The spray-dried extract is thus diluted in a mixture of water and glycerin at an amount of 1.5% by weight relative to the total weight of the water/glycerin/extract mixture and 80% of glycerin by weight relative to the total weight of the water/glycerin/extract mixture, then filtered. The extract obtained is a liquid extract.

### Example 2

### Demonstration of the Decreased Expression of Collagen XVIII over the Course of Aging of the Skin

Protocol: Samples of facial skin from 6 donors of different ages (from 10 to 69 years), referred to as “normal”, that is to say not having a disease, were collected then frozen at −80° C. The samples were cut up then fixed with a mixture of methanol/acetone during a period of 10 minutes and rinsed with PBS (phosphate-buffered saline) buffer. The samples were then incubated in the presence of a primary anti-collagen XVIII antibody (an antibody that specifically targets the collagenic part of collagen XVIII and not the endostatin part) at ambient temperature. After rinsing with PBS buffer, the samples were incubated with a secondary rabbit antibody containing a fluorescent Cy3 marker. After rinsing, the cell nuclei were stained with DAPI reagent (4′,6′-diamidino-2-phenylindole). The decrease in the protein expression of collagen XVIII present at the dermoepiderrnal junction is observed and quantified using a confocal microscope and quantified by image analysis (3 biopsies per donor (n=3) and 5 fields per biopsy). (SD: standard deviation).

Results:

TABLE 1

| | MEAN | SD |
|---|---|---|
| Donors aged 10 years | 36.81 | 11.79 |
| Donors aged 15 years | 27.07 | 4.29 |
| Donors aged 30 years | 20.60 | 8.79 |
| Donors aged 44 years | 16.51 | 3.19 |
| Donors aged 60 years | 14.26 | 2.72 |
| Donors aged 69 years | 13.34 | 3.98 |

Conclusion: The results showed a decrease in the expression of collagen XVIII with age of the donors, demonstrating the link between the expression of collagen XVIII and the aging of the skin.

### Example 3

### Increasing the Expression of Collagen XVIII in the Presence of a Khaya senegalensis Extract

### Example 3a

### Increasing the Expression of Collagen XVIII in Keratinocytes

Protocol: “Normal” human keratinocytes, that is to say not having a disease, originating from the breast of a healthy 36-year-old female donor (female, breast) were cultured in a defined medium (KSFM) during a period of 48 hours in the presence of 2 different final concentrations of the Khaya senegalensis extract prepared according to example 1a), then the culture medium was eliminated. The cell layer obtained was fixed (phosphate-buffered saline (PBS) buffer/paraformaldehyde) then permeabilized (PBS buffer/Triton).

The collagen XVIII was assayed with an anti-collagen XVIII antibody, diluted to 1/500 in a PBS buffer solution containing bovine serum albumin (BSA). After a period of 90 minutes, a secondary antibody coupled to fluorescein diluted to 1/200 is applied for a period of 2 hours in darkness.

After drying, the mixture was resolubilized with a solution of ammonium hydroxide. The fluorescence was measured (ENVision, PerkinElmer). The fluorescence results were standardized relative to the fluorescence obtained with the same cell medium in the absence of Khaya senegalensis extract (control) and were related to the cell viability obtained under each condition. The results presented correspond to the mean of 6 tests (n=6). (SD: standard deviation).

Results:

TABLE 2

| | MEAN | SD |
|---|---|---|
| Control | 100 | 14.0 |
| K. senegalensis extract 3 × 10⁻³ % (w/v medium) Ex. 1a) | 154.0 | 18.6 |
| K. senegalensis extract 1.5 × 10⁻³ % (w/v medium) | 149.9 | 11.1 |
| Ex. 1a) | | |

Conclusion: the Khaya senegalensis extract according to the invention significantly increased protein expression of collagen XVIII by human keratinocytes by at least +49% versus the untreated control (Dunnett's statistical analysis test, p<0.001).

### Example 3b

### Increasing the Expression of Collagen XVIII in Adipocytes

Protocol: Subcutaneous human pre-adipocytes that are referred to as normal, that is to say not having a disease, originating from a female donor, were cultured in a specific growth medium during a period of 3 days at a temperature of 37° C. under 5% CO₂ atmosphere. When the stage of saturation of the cell layer was reached, the medium was replaced by a differentiation medium, into which had been added the Khaya senegalensis extract according to the invention, prepared under the conditions described in example 1a), at different final concentrations in the medium (w/v), and cultured during a period of 6 days.

The protein expression of collagen XVIII was evaluated by immunocytochemistry. The above cultured cells were fixed with acetone at −20° C. for a period of 10 minutes then rinsed (PBS buffer) and placed in a serum for a period of 30 minutes at 37° C. and rinsed (PBS buffer). A primary anti-collagen XVIII antibody diluted to 1/200 was then incubated during a period of 2 hours at ambient temperature. After rinsing, a secondary antibody diluted to 1/200 was incubated during a period of 45 minutes at ambient temperature and away from light. The medium was then eliminated, then the cells were counterstained (Evans Blue) during a period of 10 minutes, then rinsed (PBS). The observations were carried out by confocal microscopy. The collagen XVIII was quantified after image analysis. The increase in the protein expression of collagen XVIII is proportional to the percentage occupation of said collagen on the images.

The results of table 3 are expressed as the mean of the percentage occupation of the collagen XVIII (n=6). (SD: standard deviation) Results:

TABLE 3

| | MEAN | SD |
|---|---|---|
| Control | 4.1 | 0.4 |
| K. senegalensis extract 1 × 10⁻⁴ % (w/v medium) Ex. 1a) | 12.9 | 0.8 |
| K. senegalensis extract 3 × 10⁻⁵ % (w/v medium) Ex. 1a) | 8.9 | 0.8 |

Conclusion: the Khaya senegalensis extract according to the invention significantly increased protein expression of collagen XVIII by human adipocytes by at least +117.1% and up to +214.6% versus the untreated control (Shapiro-Wilk statistical analysis test, p<0.001).

### Example 3c

### Increasing the Expression of Collagen XVIII in Endothelial Cells

Protocol:

Human endothelial cells that are referred to as normal, that is to say not having a disease, originating from a healthy 62-year-old donor, were cultured in a specific growth medium (EMB-2) during a period of 5 days at a temperature of 37° C. under 5% CO₂ atmosphere. The cells were then seeded on 8-well Labtecks EZ slide), still in a specific growth medium, into which had been added the Khaya senegalensis extract according to the invention, prepared under the conditions described in example 1a), at different final concentrations in the medium (w/v), and cultured during a period of 3 and 7 days.

The protein expression of collagen XVIII was evaluated by immunocytochemistry. The cultured cells below were fixed with acetone at −20° C. for a period of 10 minutes then rinsed (PBS buffer) and placed in a serum for a period of 30 minutes at 37° C. and rinsed (PBS buffer). An anti-collagen XVIII antibody diluted to 1/500 was then incubated for a period of 2 hours at ambient temperature. A secondary antibody (Anti-rabbit Alexas 488, Invitrogen) diluted to 1/250 was incubated during a period of 45 minutes at ambient temperature and away from light. The medium was then rinsed, then stained (Evans Blue) during a period of 10 minutes, then rinsed (PBS). The observation was carried out by confocal microscopy.

The quantification of the collagen XVIII was obtained after image analysis.

The increase in the protein expression of collagen XVIII is proportional to the percentage occupation of said collagen on the images.

Results:

The results obtained showed a significant increase in the expression of collagen XVIII by the endothelial cells.

### Example 4

### In Vitro Demonstration of the Improvement in the Biomechanical Properties of the Skin in the Presence of a Khaya senegalensis Extract

### Example 4a

### Improvement in the Residual Deformation of Reconstructed Skin Models

Protocol: During each step of renewing the medium, the Khaya senegalensis extract according to the invention, prepared under the conditions described in example 1a), was added at a concentration of 0.00075% (w/v). Human keratinocytes that are referred to as “normal”, that is to say not having a disease, obtained from a breast biopsy from a healthy 30-year-old female donor, and human fibroblasts that are referred to as “normal”, that is to say not having a disease, obtained from a breast biopsy from a healthy 18-year-old female donor, were obtained. The fibroblasts were cultured in a specific DMEM (Dulbecco's modified Eagle's medium) medium containing 10% calf serum and antibiotics (Normocin, 100 μg/ml). The keratinocytes were cultured in a K-FSM medium (Life Technologies) containing antibiotics, until sub-confluence. Dermal fibroblasts were seeded onto a substrate consisting of collagen, chitosan and glycosaminoglycans, and these dermis equivalents were cultured during a period of 28 days at a temperature of 37° C. under 5% CO₂ atmosphere. The medium containing the fibroblasts cultured on specific DMEM medium was supplemented with 10% of fetal calf serum, ascorbic acid (50 μg/ml) and antibiotics.

The keratinocytes were then seeded on dermis equivalents and were cultured during an additional period of 21 days in a simple DMEM medium containing bovine serum albumin (BSA) (0.8%), insulin, hydrocortisone, ascorbic acid and antibiotics. In order to trigger the differentiation process, the reconstructed skins were placed at an air-liquid interface 7 days after the seeding of the keratinocytes.

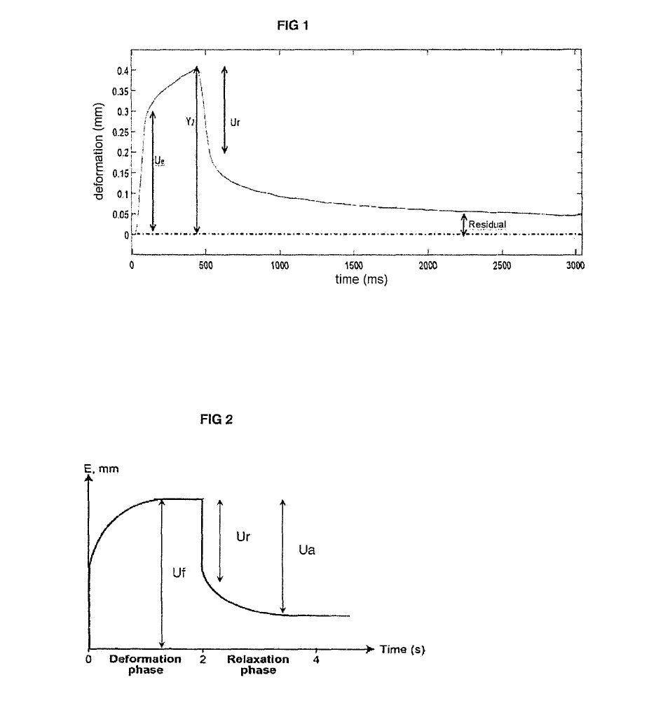

The biomechanical properties of the reconstructed skin models were measured via a method based on evaluating the deformation of the skin under a controlled air stream. The deformation of the skin is monitored by virtue of a laser coupled to the equipment. A deformation curve is then obtained (cf. FIG. 1). The “residual” value reflects the residual deformation of the skin in mm (Residual, FIG. 1). The smaller this “residual” coefficient is, the more the skin is elastic and resumes its initial form. The results are expressed in mm (n=13) (SD: standard deviation). Results:

TABLE 4

| | Residual | |
|---|---|---|
| | (mm) | SD |
| Control | 0.0451 | 0.0131 |
| Khaya senegalensis extract 7.5 × 10⁻⁴ % Ex. 1a) | 0.0318 | 0.0189 |

Conclusion: the results showed that reconstructed skins cultured in the presence of the Khaya senegalensis extract according to the invention have a significantly lower residual deformation rate than when they are cultured in the absence of the extract (Mann-Whitney statistical analysis test p<0.05). The extract according to the invention therefore does indeed make it possible to increase the elasticity of the skin.

### Example 4b

### Improvement of the Viscoelastic Component of Reconstructed Skins in the Presence of the Khaya senegalensis Extract

Protocol: The reconstructed skins were cultured under the conditions described above. The evaluation of the viscoelastic component of the skins is reflected via the parameter (Y2−Ue)/Y2 (FIG. 1). When said parameter decreases, the elasticity of the skin increases, implying that the skin returns more easily to its initial state. The results are presented in table 5 and are expressed by the mean of 13 tests (n=13).

Results:

TABLE 5

| | MEAN | SD |
|---|---|---|
| Control | 0.333 | 0.031 |
| Khaya senegalensis extract 7.5 × 10⁻⁴ % Ex. 1a) | 0.256 | 0.062 |

Conclusion: the results showed a significant decrease (Mann-Whitney statistical analysis test p<0.001) of the parameter (Y2−Ue)/Y2 by at least 23.1% relative to the control in reconstructed skins cultured in the presence of the Khaya senegalensis extract according to the invention, reflecting an increase in the elasticity of the reconstructed skins.

### Example 5

### In Vivo Demonstration of the Effects on the Skin of a Khaya senegalensis Extract

Protocol: The cream described in example 6 comprising the Khaya senegalensis extract prepared according to example 1e) at a final concentration by weight of 1% relative to the total weight of the cream, and the same placebo cream without extract, were applied every day each to a half face of 25 female Caucasian volunteers aged from 53 to 65 years, having skin with visible pores and having a loss of elasticity, during a period of 2 months.

### Example 5a

### In Vivo Measurement of the Skin Elasticity

The skin elasticity was evaluated by cutometry, taking into account the R2 and R7 coefficients. The R2 coefficient corresponds to the Ua/Uf ratio (part between the maximum amplitude and the capacity for deformation of the skin) (cf. FIG. 2), reflecting overall elasticity. The R7 coefficient corresponds to the Ur/Uf ratio, reflecting the immediate elasticity of the skin (FIG. 2). The results are expressed as % increase in elasticity at the times T28 and T56 days, compared to the control (cream without K. senegalensis extract).

Results:

TABLE 6
R2 coefficient (Ua/Uf)

| | % improvement vs placebo |
|---|---|
| | Ua/Uf coefficient (R2) |
| | T56 |
| 1% K. senegalensis extract (w/w) | +10.70% |

Conclusion: The results showed a significant increase (p<0.05, Student's t test) in the overall elasticity of the skin of the half faces treated with the cream comprising the Khaya senegalensis extract after 56 days of daily application, compared to the skin of the half faces treated with the placebo cream.

TABLE 7
R7 coefficient (Ur/Uf)

% improvement vs placebo
Ur/Uf coefficient (R7)

| | T28 | T56 |
|---|---|---|
| 1% K. senegalensis extract (w/w) | +9.60 | +11.60 |

Conclusion: The results showed a significant increase (p<0.05, Student's t test) of 9.6% in the immediate elasticity of the skin of the half faces treated with the cream comprising the Khaya senegalensis extract after 28 days of daily application, compared to the skin of the half faces treated with the placebo cream, and of 11.60% in the immediate elasticity of the skin of the half faces treated with the cream comprising the Khaya senegalensis extract after 56 days of daily application, compared to the skin of the half faces treated with the placebo cream.

### Example 5b

### In Vivo Measurement of the Density of the Cutaneous Pores

The pore density was evaluated by the fringe projection method, by analyzing the cutaneous microrelief via the parameter referred to as “curvature”. The larger this parameter is, the greater the surface homogeneity (pores and cutaneous microrelief). Since this measurement was carried out on the cheeks, it can be directly correlated to a measurement of the pore density. A decrease in this parameter therefore reflects a decrease in the density of the cutaneous pores and of the cutaneous microrelief.

Results: The results are expressed as % reduction in the curvature parameter compared to the placebo.

TABLE 8

| | T28 | T56 |
|---|---|---|
| 1% K. senegalensis extract (w/w) | −22.8% | −38.2% |

Conclusion: The results showed a decrease in the parameter of interest, reflecting a significant decrease (p<0.05, Student's t test) in the pore density of 22.80% in the presence of the cream comprising the K. senegalensis extract according to the invention after 28 days of application.

### Example 5c

### In Vivo Measurement of Skin Wrinkles

The effectiveness of the extract according to the invention on crow's feet wrinkles was measured at the time T28 (28 days) by measuring the volume of said wrinkles by the fringe projection method under the conditions already described above (protocol for example 5b). The results are expressed as % reduction in the volume of the wrinkles vs. control (cream without extract), in the presence of the cream comprising the K. senegalensis extract according to the invention.

Results:

TABLE 9

| | T56 |
|---|---|
| 1% K. senegalensis extract (w/w) | −16.2 |

Conclusion: The K. senegalensis extract decreased the volume of the wrinkles detected at the time T56 days by 16.2% compared to the control (p<0.05, Student's t test).

### Example 6

### Example of Cosmetic Composition Comprising a Khava senegalensis Extract

Methods known to those skilled in the art are used to mix together the different phases A, B, C and D below in order to prepare a composition according to the present invention. The proportions are expressed as %.

Phase A

| | |
|---|---|
| Glyceryl stearate, PEG-100 Stearate | 4.00 |
| Pentaerythrityl distearate | 1.50 |
| Cetearyl Isononanoate | 3.00 |
| Propylheptyl caprylate | 5.00 |
| Coco caprylate | 2.00 |
| Dicaprylyl carbonate | 3.00 |
| Dimethicone | 1.00 |

Phase B

| | |
|---|---|
| Water | 64.43 |
| Propylene glycol, phenoxyethanol, chlorphenesin, methylparaben | 1.00 |
| Glycerin | 1.57 |
| Xanthan gum | 0.20 |
| Butylene glycol | 2.00 |
| Sodium hydroxide, water | 0.15 |

Phase C

| | |
|---|---|
| Carbomer | 0.15 |
| Water | 10 |

Phase D

| | |
|---|---|
| Khaya senegalensis extract obtained according to example 1 e) | 1.00 |

1-14. (canceled)

15. A cosmetic method for maintaining and/or increasing the expression of collagen XVIII in the skin and/or the mucous membranes and/or the scalp and/or the skin appendages comprising administering to a patient in need thereof of an effective amount of a Khaya senegalensis extract obtained by aqueous extraction.

16. The method as claimed in claim 15, for maintaining and/or increasing the expression of collagen XVIII in the dermoepidermal junction of the skin, and/or the mucous membranes and/or the scalp and/or the sebaceous glands, and/or the basal laminae of adipocytes, endothelial cells, sudoriparous glands and/or hair follicles.

17. The method as claimed in claim 15, wherein the expression of collagen XVIII is protein expression.

18. The method as claimed in claim 15, wherein the collagen XVIII is expressed by keratinocytes, adipocytes, endothelial cells, epithelial cells of the sudoriparous glands and/or stern cells of hair follicles.

19. The method as claimed in claim 15 for decreasing the visibility of cutaneous pores and/or making the skin smoother and/or for maintaining and/or increasing the firmness and/or elasticity of the skin and/or the mucous membranes and/or the scalp and/or for limiting and/or reducing perspiration and/or limiting and/or reducing the loss of head hair and/or body hair and/or increasing the growth of head hair and/or body hair and/or reducing or limiting the production of sebum.

20. The method as claimed in claim 15, wherein the Khaya senegalensis extract is a bark extract.

21. The method as claimed in claim 15, wherein the extract is applied topically.

22. The method as claimed in claim 15, wherein the Khaya senegalensis extract is present in a cosmetic composition comprising at least one cosmetically acceptable excipient at a concentration from 1×10⁻⁴% to 10% by weight relative to the total weight of the composition.

23. The method as claimed in claim 21, wherein the extract is applied onto the skin and/or the mucous membranes and/or the scalp.

24. The method as claimed in claim 23, wherein the topical application of the Khaya senegalensis extract obtained by aqueous extraction is carried out over all or part of the body and/or the face and/or the scalp.

25. The method as claimed in claim 22, wherein the cosmetic composition comprises the Khaya senegalensis extract according to the invention at a concentration from 1×10⁻⁴% to 5% by weight relative to the total weight of the composition.

26. A method for preventing and/or treating diseases involving a loss of gene and/or protein expression of type XVIII collagen comprising the administration to a patient in need thereof of an effective amount of a Khaya senegalensis extract.

27. The method as claimed in claim 26, wherein the extract is in the form of a dermatological and/or pharmaceutical composition comprising at least one dermatologically and/or pharmaceutically acceptable excipient.

28. The method as claimed in claim 27, wherein the extract is present in the pharmaceutical, composition at a concentration from 1×10⁻⁴% to 10% by weight, relative to the total weight of the composition.

29. The method as claimed in claim 24, wherein the topical application of the Khaya senegalensis extract obtained by aqueous extraction is carried out over all or part of the legs, thighs, arms, stomach, bust, neck, armpits and/or the lips.

30. The method as claimed in claim 24, wherein the topical application of the Khaya senegalensis extract obtained by aqueous extraction is carried out over all or part of the cheeks, forehead, chin, lips and/or the area around the eyes.

31. The method as claimed in claim 24, wherein the topical application of the Khaya senegalensis extract obtained by aqueous extraction is carried out over all or part of the “T” zone of the face.

32. The method as claimed in claim 25, wherein the cosmetic composition comprises the Khaya senegalensis extract according to the invention at a concentration from 1×10⁻³% to 3% by weight relative to the total weight of the composition.

33. The method as claimed in claim 32, wherein the cosmetic composition comprises the Khaya senegalensis extract according to the invention at a concentration from 0.001% to 0.1% by weight relative to the total weight of the composition.

34. The method as claimed in claim 26, wherein the diseases involving a loss of gene and/or protein expression of type XVIII collagen are chosen in the group consisting of the diseases involved in eye development, the diseases involved in cerebral development, the diseases involved in the nervous system and certain glomerulopathies.
